(19) United States
(12) Patent Application Publication
(10) Pub. No.: US 2024/0407992 A1
(43) Pub. Date: Dec. 12, 2024

(54) MODIFIED SOPHOROLIPIDS FOR CONDITIONING OF HAIR AND TEXTILES

(71) Applicant: LOCUS SOLUTIONS IPCO, LLC, SOLON, OH (US)

(72) Inventors: LEE SPEIGHT, SOLON, OH (US); DANIEL HAGAMAN, MECHANICSVILLE, VA (US); ANDREW MORRIS, RICHMOND, VA (US); AYENDA ZEMAITIS, MIDLOTHIAN, VA (US)

(21) Appl. No.: 18/249,246

(22) PCT Filed: Feb. 28, 2023
(86) PCT No.: PCT/US2023/014017

§ 371 Date: Apr. 15, 2023

Related U.S. Application Data
(60) Provisional application No. 63/314,757, filed on Feb. 28, 2022.

(51) Int. Cl. A61K 8/60; A61Q 5/12; C11D 1/58

(52) U.S. Cl. CPC A61K 8/602; A61Q 5/12; C11D 1/58

(57) ABSTRACT

The subject invention provides conditioning compositions for hair, fibers and textiles, wherein the conditioning compositions comprise derivatized cationic biosurfactants.

### CROSS-REFERENCE TO RELATED APPLICATION

This application claims priority to U.S. Provisional Patent Application No. 63/314,757, filed Feb. 28, 2022, which is incorporated herein by reference in its entirety.

### BACKGROUND

Consumers utilize, and are exposed to, household and personal care products every day. For example, most consumers' daily routine includes the use of hair care, make-up, cleansers, laundry detergents, and others. While many of these types of products contain harsh chemicals as active ingredients, additional chemicals may be included as additives that, for example, help with properties such as viscosity, foaming, and solubility of fragrances, dyes, and active components.

One specific category of products of interest is hair care products. Shampooing of hair cleans the hair by removing excessive environmental soils and sebum. Shampooing can also result in tangled and unmanageable hair, especially with longer length hair. Furthermore, once the hair dries, it is often left in a dry, rough, staticky, lusterless and/or frizzy condition due to the removal of the hair's natural oils and other natural conditioning and moisturizing components.

To avoid these problems, hair conditioning compositions are typically applied to the hair immediately after shampooing and rinsing. The conditioning composition is worked through the hair and may be then used as a leave-on conditioner or it can be rinsed from the hair with water. Similar types of compositions have also been used in the conditioning of textiles and fibers.

Traditionally, hair and fiber conditioning compositions have utilized cationic surfactants. Cationic surfactants are those in which the surfactant activity resides in the positively charged cation portion of the molecule. The cationic surfactants are therefore attracted to the negatively charged hair or fiber surface and deposited on the hair or fiber. Among cationic surfactants, quaternary ammonium compounds (QACs) are particularly suited to the treatment hairs and fibers. Thus, many conditioning products are based on quaternary ammonium compounds, such as stearyl trimethylammonium chloride, behenyl trimethylammonium chloride and distearyl dimethylammonium chloride.

Toxicity to humans and domestic animals is the short-term problem with existing personal care and household products. The environmental damage that can be attributed to these ingredients has yet to be fully understood; however, it is largely dose dependent. In some instances, QACs have been shown to persist in the environment. While biodegradation pathways have been shown under laboratory aerobic conditions, QACs, especially those containing aromatic scaffolds, are prone to accumulate in environmental sludges and partition poorly to aqueous media. This removes QACs from an aerobic environment in which biodegradation can occur. Accumulation of QACs in anaerobic environmental sludges and soils poses a significant challenge. Typical methods to remove other nitrogen-containing environmental contaminations, both natural and human-activity promoted, are severely limited by the presence of QACs.

QAC biodegradation in anaerobic sludge environments is possible, but the principal breakdown products of QACs include short alkyl amines such as methyl amine. Alkyl amines can accumulate within the microbes capable of degrading QACs, leading to inhibition of QAC degrading enzymes and/or toxicity to the microbes themselves. Further complicating the matter, QACs have been shown to inhibit methanogenesis and other anaerobic digestion pathways used by microbes to degrade other compounds. The risk of QACs in the environment thus entails the risk of accumulation of other hazardous chemicals that would typically degrade.

In addition to the problem of QAC accumulation in the environment and its impacts on microbes essential for biodegradation pathways, recent research has suggested that QAC environmental accumulation is accelerating the development of microbes that are resistant to traditional antibiotics. The same genes found to be responsible for conferring resistance to QACs in bacteria are associated with drug resistant bacteria studied by medical researchers. The mechanism of this resistance involves the use of efflux proteins with broad specificity towards exogenous compounds. Thus, use, and environmental accumulation of, QACs will lead to the selection of bacteria that are capable of resisting them, and, inadvertently, potentially resist traditional antibiotics.

There are a variety of nature-derived substances that have been shown to have some efficacy as conditioning ingredients. One such substance is palm oil and its derivatives (e.g., palmitate, glyceryl, stearic acid, sodium laureth sulfate, sodium lauryl sulfate). In shampoos and conditioners, palm oil is used to help restore the natural oils of the hair that are stripped away by other cleansing chemicals present in most shampoos.

While production and harvesting of palm oil is considered low cost economically, the environmental costs are becoming increasingly worrying. Mass-production of palm oil is causing destruction of rainforests, endangerment to native species, and water pollution due to mishandling of processing effluents.

Increasingly, consumers are looking for household and personal care products that are non-toxic, non-irritating to the skin and/or eyes, and with a reduced impact on the environment, but these safer and more sustainable products are still expected to deliver performance on many attributes, such as conditioning of hair and fibers, at parity to traditional products. Due to the limited set of natural or sustainable materials that meet these needs, formulating safe and environmentally friendly conditioning compositions remains a challenge.

Thus, there is a need for improved compositions that are effective for imparting the beneficial effects of traditional conditioners on hair and textiles, and that do not contain harmful or polluting chemicals or synthetically derived ingredients.

### BRIEF SUMMARY

The present invention provides microbe-based products, as well as methods of their use in the conditioning of hair, fibers and textiles. More specifically, the present invention provides microbial-derived ingredients for use in formulating hair care products, household laundry products, and textile processing materials. Advantageously, in certain embodiments, the microbial-derived ingredients can be useful for replacing and/or reducing the use of traditional conditioning compounds, such as QACs and palm oil.

In preferred embodiments, the present invention provides microbial-derived surfactants, or biosurfactants, for use in conditioning formulations for hair, laundry, and textile fibers. In certain specific embodiments, the biosurfactant has been derivatized and/or purified to produce and/or enhance one or more desired functionalities, preferably, conditioning functionalities.

In certain embodiments, the conditioning functionalities include one or more of the following non-limiting examples: reducing static and/or frizz; increasing strength; reducing breakage; reducing tangling and matting; improving shine and softness; increasing oil and/or moisture retention; reducing wrinkles; and/or imparting a fragrance.

In certain embodiments, the biosurfactant is a glycolipid selected from, for example, sophorolipids (SLP), mannosylerythritol lipids (MEL), rhamnolipids (RLP) and trehalose lipids (TL).

In certain specific embodiments, the biosurfactant comprises a sophorolipid, or a mixture of SLP molecules comprising, for example, lactonic SLP, linear SLP, de-acetylated SLP, mono-acetylated SLP, di-acetylated SLP, esterified SLP, SLP with varying hydrophobic chain lengths, SLP with fatty acid-amino acid complexes attached, and others, including those that are and/or are not specifically exemplified within this disclosure.

In a specific preferred embodiment, the biosurfactant is a derivatized SLP, or a mixture of derivatized SLP, wherein the molecular structure of the SLP molecule(s) has been altered to produce a linear (acidic) cationic SLP derivative. More specifically, in preferred embodiments, the linear cationic SLP derivative has been altered to comprise a cationic amino acid residue, such as, e.g., arginine, lysine or histidine; a peptide containing repeats of arginine, lysine and/or histidine; or a peptide containing glycine spacers between individual amino acids and/or between the SLP scaffold and an amino acid or peptide residue.

In certain embodiments, the derivatized cationic SLP according to the present invention can be used as active ingredients in environmentally friendly conditioning compositions for hair, fibers and textiles. Advantageously, in preferred embodiments, the compositions and methods are at least as effective conditioners as QACs, and other synthetic cationic conditioning ingredients, as well as palm oil.

In certain specific embodiments, the conditioning composition can further comprise additional biosurfactants. For example, in some embodiments, the conditioning composition comprises a derivatized linear cationic SLP and one or more other SLP molecules that have not been derivatized according to the present invention. In other embodiments, the conditioning composition comprises a derivatized linear cationic SLP and one or more MEL molecules. In yet another exemplary embodiment, the conditioning composition comprises a derivatized linear cationic SLP, one or more other SLP molecules, and one more MEI, molecules.

Optionally, the conditioning composition can further comprise one or more other components, including, for example, carriers (e.g., water), solvents, organic and/or inorganic acids, essential oils, botanical extracts, cross-linking agents, chelators, fatty acids, alcohols, pH adjusting agents, reducing agents, buffers, enzymes, dyes, colorants, fragrances, preservatives, emulsifiers, foaming agents, bleaching agents, polymers, thickeners and/or viscosity modifiers.

In some embodiments, the conditioning compositions can be used in a hair care product, such as, for example, a shampoo, a conditioner or cream rinse, a de-tangler, or a styling product. In some embodiments, the conditioning composition can be used in a household or industrial laundry product, such as a fabric softener or a dryer sheet. In some embodiments, the conditioning composition can be used in a treatment for the conditioning of raw fibers or fabrics prior to the assembly of garments, rugs and other textiles.

In preferred embodiments, the subject invention further provides a method for conditioning hair, fibers or textiles, wherein the method comprises contacting a conditioning composition of the present invention with the hair, fibers or textiles for an amount of time sufficient to impart a conditioning effect thereon. In some embodiments, the composition is applied in the presence of water or another solvent. In some embodiments, the composition is rinsed from the hair, fibers or textiles after being contacted therewith, while in other embodiments, the composition left in place without rinsing.

Advantageously, the present methods are safe for use in, for example, household, commercial, healthcare and industrial settings and in the presence of humans, plants and animals. Additionally, the conditioning compositions of the present invention can be produced and used without causing harm to users and without releasing large quantities of polluting and toxic compounds into the environment. Furthermore, the compositions and methods utilize components that are biodegradable and toxicologically safe. Thus, the present invention can be used in a variety of industries as a “green” conditioning ingredient.

### DETAILED DESCRIPTION

The present invention provides microbe-based products, as well as methods of their use in the conditioning of hair, fibers and textiles. More specifically, the present invention provides microbial-derived ingredients for use in formulating hair care products, household laundry products, and textile processing materials. Advantageously, in certain embodiments, the microbial-derived ingredients can be useful for replacing and/or reducing the use of traditional conditioning compounds, such as QACs and palm oil.

In preferred embodiments, the present invention provides microbial-derived surfactants, or biosurfactants, for use in conditioning formulations for hair, laundry, and textile fibers. Biosurfactants are amphiphilic molecules consisting of both hydrophobic (e.g., a fatty acid) and hydrophilic domains (e.g., a sugar). Due to their amphiphilic nature, biosurfactants can partition at the interfaces between different fluid phases such as oil/water or water/air interfaces. Unlike synthetic surfactants, biosurfactants can be effective in hot or cold water, and at either extreme of the pH scale. Additionally. biosurfactants are biodegradable and non-toxic.

Glycolipid biosurfactants, in particular, have many important physiological roles in cellular biology, chiefly as a major component of cell membranes; however, they have gained attention in recent years due to potentially servicing as biological replacements for legacy surfactants.

In certain embodiments of the present invention, sophorolipids (SLP) are specific glycolipids of interest. Sophorolipids are glycolipid biosurfactants produced by, for example, various yeasts of the Starmerella clade. SLP consist of a disaccharide sophorose linked to long chain hydroxy fatty acids. They can comprise a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid can have, for example, 11 to 20 carbon atoms, and may contain one or more unsaturated bonds. Furthermore, the sophorose residue can be acetylated on the 6- and/or 6′-position(s). The fatty acid carboxyl group can be free (acidic or linear form) or internally esterified at the 4″-position (lactonic form). In most cases, fermentation of SLP results in a mixture of hydrophobic (water-insoluble) SLP, including, e.g., lactonic SLP, mono-acetylated linear SLP and di-acetylated linear SLP, and hydrophilic (water-soluble) SLP, including, e.g., non-acetylated linear SLP.

As used herein, the term “sophorolipid,” “sophorolipid molecule,” “SLP” or “SLP molecule” includes all forms, and isomers thereof, of SLP molecules, including, for example, acidic (linear) SLP and lactonic SLP. Further included are mono-acetylated SLP, di-acetylated SLP, esterified SLP, SLP with varying hydrophobic chain lengths, SLP with fatty acid-amino acid complexes attached, and other, including those that are and/or are not described within in this disclosure.

In some embodiments, SLP molecules can be represented by General Formula (1) and/or General Formula (2), and are obtained as a collection of 30 or more types of structural homologues having different fatty acid chain lengths (R³), and, in some instances, having an acetylation or protonation at R¹ and/or R².

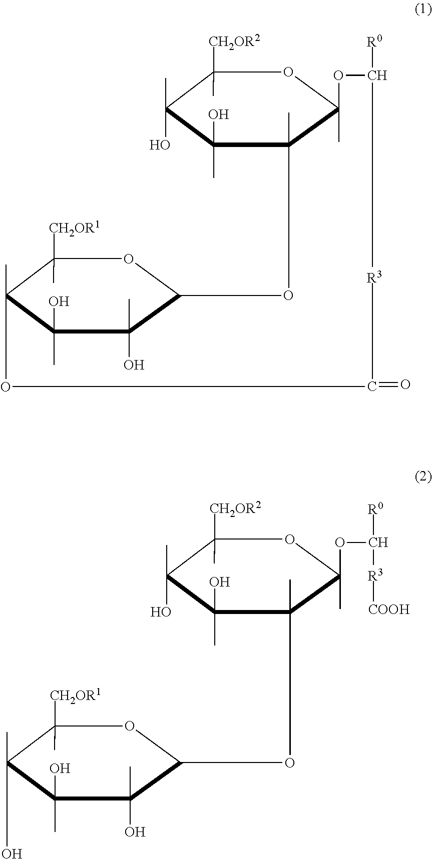

In General Formula (1) or (2), R⁰ can be either a hydrogen atom or a methyl group. R¹ and R² are each independently a hydrogen atom or an acetyl group. R³ is a saturated aliphatic hydrocarbon chain, or an unsaturated aliphatic hydrocarbon chain having at least one double bond, and may have one or more Substituents.

Non-limiting examples of the Substituents include halogen atoms, hydroxyl, lower (C1-6) alkyl groups, halo lower (C1-6) alkyl groups, hydroxy lower (C1-6) alkyl groups, halo lower (C1-6) alkoxy groups, and others, such as those that are described within the present disclosure. R³ can have, for example, 11 to 20 carbon atoms.

Fermentation of yeast cells in a culture substrate including a sugar and/or lipids and fatty acids with carbon chains of differing length can be used to produce a variety of SLP. The yeast Starmerella (Candida) bombicola is one of the most widely recognized producers of SLP. Typically, the yeast produces both lactonic and linear SLP during fermentation, with about 60-70% of the SLP comprising lactonic forms, and the remainder comprising lactonic forms.

### Selected Definitions

As used herein, the term “conditioning” refers to one or more of the following non-limiting examples as it is applicable to hair, fibers and/or textiles: reducing static and/or frizz; increasing strength; reducing breakage; reducing tangling and matting; improving shine and softness; increasing oil and/or moisture retention; reducing wrinkles; and/or imparting a fragrance.

As used herein, “dermatologically-acceptable,” “cosmetically-acceptable” and “topically-acceptable” are used interchangeably and are intended to mean that a particular component is safe and non-toxic for application to the integument (e.g., skin, scalp) at the levels employed. In one embodiment, the components of the composition are recognized as being Generally Regarded as Safe (GRAS).

As used herein, the term “effective amount” refers to an amount of something (e.g., a compound, a composition, time) that is capable of achieving a desired amount of conditioning in hair, fibers and/or textiles. The actual amount will vary depending on a number of factors including, but not limited to, the amount of conditioning required, the type of hair, fiber and/or textile, and the manner of administration.

As used herein, a “microbe-based composition” means a composition that comprises components that were produced as the result of the growth of microorganisms or other cell cultures. Thus, the microbe-based composition may comprise the microbes themselves and/or by-products of microbial growth (e.g., biosurfactants, solvents and/or enzymes). The cells may be in a vegetative state or in spore form, or a mixture of both. The cells may be planktonic or in a biofilm form, or a mixture of both. The cells may be live or inactive, intact or lysed. The cells can be removed from the medium in which they were grown, or present at, for example, a concentration of at least 1×10³, 1×10⁴, 1×10⁵, 1×10⁶, 1×10⁷, 1×10⁸, 1×10⁹, 1×10¹⁰, or 1×10¹¹ or more cells per milliliter of the composition. In one embodiment, the microbe-based composition may comprise only the medium in which the cells were grown, with the cells removed (although, in some instances, some residual cellular matter may also remain in the medium). The by-products of growth may be present in the medium and can include, for example, metabolites, cell membrane components, expressed proteins, and/or other cellular components. In one embodiment, the microbe-based composition comprises only microbial growth by-products.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, carriers, and other additives and/or adjuvants suitable for a particular application. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

A “metabolite” refers to any substance produced by metabolism (e.g., a growth by-product) or a substance necessary for taking part in a particular metabolic process. Examples of metabolites include, but are not limited to, enzymes, acids, solvents, alcohols, proteins, carbohydrates, vitamins, minerals, microelements, amino acids, polymers, and biosurfactants.

As used herein, the terms “isolated” or “purified,” when used in connection with biological or natural materials such as nucleic acid molecules, polynucleotides, polypeptides, proteins, organic compounds, such as small molecules, microorganism cells/strains, or host cells, means the material is substantially free of other compounds, such as cellular material, with which it is associated in nature. That is, the materials do not occur naturally without these other compounds and/or have different or distinctive characteristics compared with those found in the native material.

In certain embodiments, purified compounds are at least 60% by weight the compound of interest. Preferably, the preparation is at least 75%, more preferably at least 90%, and most preferably at least 99% or 100% (w/w) of the desired compound by weight. Purity is measured by any appropriate standard method, for example, by column chromatography, thin layer chromatography, or high-performance liquid chromatography (HPLC) analysis.

As used herein, an “isomer” refers to a molecule with an identical chemical formula to another molecule but having unique structures. Isomers can be constitutional isomers, where atoms and functional groups are bonded at different locations, and stereoisomers (spatial isomers), where the bond structure is the same but the geometrical positioning of atoms and functional groups in space is different. MEL isomers, for example, can differ in bond type and bond location of the carbohydrate, fatty acid and/or acetyl groups.

As used herein, the term “subject” refers to an animal, preferably a mammal. The preferred subject in the context of this invention is a human. The subject can be of any gender and any age or stage of development including infant, toddler, adolescent, teenager, young adult, middle-aged, or senior.

As used herein, “surfactant” means a surface-active substance, or a compound that lowers the surface tension (or interfacial tension) between two phases. Surfactants act as, e.g., detergents, wetting agents, emulsifiers, foaming agents, and/or dispersants. By “biosurfactant” is meant a surface active agent produced by a living organism, and/or produced using naturally-derived substrates.

In general, “textile” refers to any flexible material, such as a fabric, cloth or carpet, created by interlocking yarns or threads, which are produced by spinning raw fibers into long and twisted lengths. The interlocking can be achieved via, for example, weaving, knitting, crocheting, knotting, tatting, felting, bonding or braiding. In addition to the flexible material, “textile” as used herein can also include the finished products created using the flexible material, as well as the raw materials involved in producing the flexible materials, including the raw fibers, yarns and threads. In certain embodiments, finished textiles include clothing, upholstery, drapes, carpets and rugs. In certain embodiments, textiles can also include paper products.

Textiles can be made from, for example, protein-rich sources (e.g., wool, silk, hair, fur), cellulose-rich sources (e.g., cotton, flax, hemp, coconut, wood, and other plants), minerals (e.g., asbestos), as well as synthetic sources (e.g., polyamines, polyesters, acrylonitriles, polyurethanes, and other polymers).

As used herein, “topical” means suitable for local application externally to the skin, scalp, or hair. In other words, a topical composition is not intended for application to a subject via oral, intravenous, intramuscular, intrathecal, subcutaneous, sublingual, buccal, rectal, vaginal, inhalation, ocular or otic routes.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example, within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein. Other features and advantages of the invention will be apparent from the following description of the preferred embodiments thereof, and from the claims. All references cited herein are hereby incorporated by reference.

### Conditioning Compositions

The present invention provides microbe-based products, as well as methods of their use in the conditioning of hair, fibers and textiles. More specifically, the present invention provides microbial-derived ingredients for use in formulating hair care products, household laundry products, and textile processing materials. Advantageously, in certain embodiments, the microbial-derived ingredients can be useful for replacing and/or reducing the use of traditional conditioning compounds, such as QACs and palm oil.

In some embodiments, the conditioning compositions can be used in a hair care product, such as, for example, a shampoo, a conditioner or cream rinse, a de-tangler, or a styling product. In some embodiments, the conditioning composition can be used in a household or industrial laundry product, such as a fabric softener or a dryer sheet. In some embodiments, the conditioning composition can be used in a treatment for the conditioning of raw fibers or fabrics prior to the assembly of garments, fabrics, rugs and other textiles.

In preferred embodiments, the present invention provides microbial-derived surfactants, or biosurfactants, for use in conditioning formulations for hair, laundry, and textile fibers. In certain specific embodiments, the biosurfactant has been derivatized and/or purified to produce and/or enhance one or more desired functionalities, preferably, conditioning functionalities.

In certain embodiments, the biosurfactant is a glycolipid selected from, for example, sophorolipids (SLP), mannosylerythritol lipids (MEL), rhamnolipids (RLP) and trehalose lipids (TL).

In some embodiments, the biosurfactants are utilized in a crude form, wherein a biosurfactant molecule is present in the growth medium (e.g., broth) in which a biosurfactant-producing microorganism is cultivated and is collected therefrom without purification. The crude form can comprise, for example, at least 0.001%, 1%, 5%, 10%, 20%, 30%, 40%, 50%, 60%, 70%, 80%, 90% or 99% amphiphilic molecules in the growth medium. In alternate embodiments, the biosurfactant is extracted from the growth medium and, optionally, derivatized and/or purified.

In certain specific embodiments, the biosurfactant comprises a sophorolipid, or a mixture of SLP molecules comprising, for example, lactonic SIP, linear SLP, de-acetylated SLP, mono-acetylated SLP, di-acetylated SLP, esterified SLP. SLP with varying hydrophobic chain lengths, SLP with fatty acid-amino acid complexes attached, and others, including those that are and/or are not specifically exemplified within this disclosure.

In a specific preferred embodiment, the biosurfactant is a derivatized SLP, or a mixture of derivatized SLP, wherein the molecular structure of the SLP molecule(s) has been altered to produce a linear (acidic) cationic SLP derivative. More specifically, in preferred embodiments, the linear cationic SLP derivative has been altered to comprise a cationic amino acid residue, such as, e.g., arginine, lysine or histidine; a peptide containing repeats of arginine, lysine and/or histidine; or a peptide containing glycine spacers between individual amino acids and/or between the SLP scaffold and an amino acid or peptide residue.

In certain embodiments, the derivatized SLP has a formula according to General Formulas (3), (4), (5), or (6), wherein R=lysine, histidine, glyine, arginine, or peptides comprising repeats of lysine, histidine and/or arginine and R₁=H, acetyl, butyl, or isobutyl

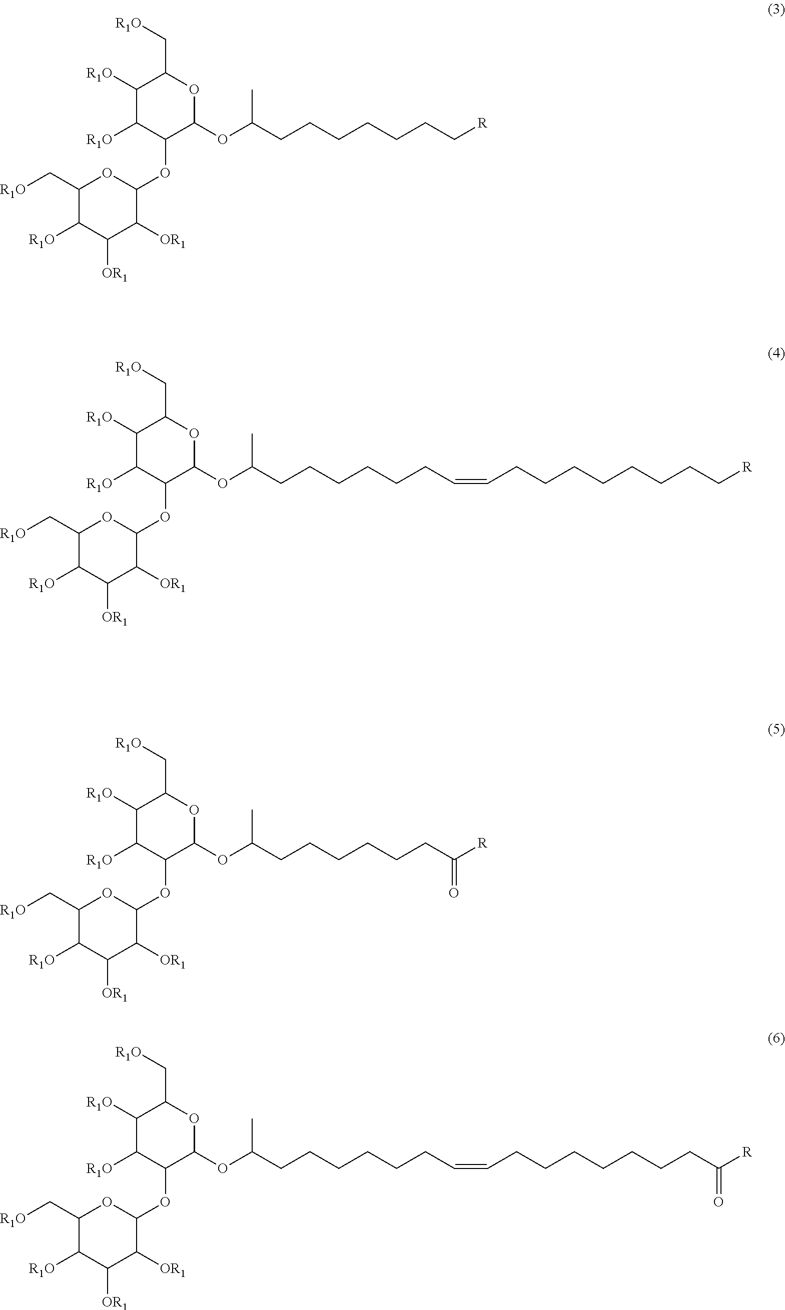

In certain embodiments, the conditioning composition comprises the derivatized SLP of General Formulas (3)-(6) at of dosing rate of about 100 ppm to about 250,000 ppm, about 200 ppm to about 100,000 ppm, about 300 ppm to about 75,000 ppm, about 400 ppm to about 50,000 ppm, or about 500 ppm to about 25,000 ppm.

In certain embodiments, the conditioning composition comprises the derivatized SLP at of dosing rate of about 0.01 wt % to about 25 wt %, about 0.05 wt % to about 20 wt %, about 0.1 wt % to about 15 wt %, or about 0.5 wt % to about 10 wt %.

In certain specific embodiments, the conditioning composition can further comprise additional biosurfactants. For example, in some embodiments, the conditioning composition comprises a derivatized linear cationic SLP and one or more other SLP molecules that have not been derivatized according to the present invention.

The mixture ratio of the cationic derivatized SLP to the non-derivatized SLP can range from 1:1 to 1:1000, from 1:5 to 1:500, or from 1:10 to 1:100.

In other embodiments, the conditioning composition comprises a derivatized linear cationic SLP and one or more mannosylerythritol lipid (MEL) molecules.

The mixture ratio of the cationic derivatized SLP to the MEL can range from 1:1 to 1:1000, from 1:5 to 1:500, from 1:10 to 1:100, from 1000:1 to 1:1, from 500:1 to 5:1, or from 100:1 to 10:1.

In yet another exemplary embodiment, the conditioning composition comprises a derivatized linear cationic SLP, one or more other SLP molecules, and one or more MEL molecules.

In one embodiment, MEL comprise either 4-O-B-D-mannopyranosyl-meso-erythritol or 1-O-B-D-mannopyranosyl-meso-erythritol as the hydrophilic moiety, and fatty acid groups and/or acetyl groups as the hydrophobic moiety. One or two of the hydroxyls, typically at the C4 and/or C6 of the mannose residue, can be acetylated. Furthermore, there can be one to three esterified fatty acids, from 8 to 12 carbons or more in chain length.

MEL and MEL-like substances (e.g., mannose-based substances) are produced mainly by Pseudozyma spp. (e.g., P. aphidis) and Ustilago spp. (e.g., U. maydis), with significant variability among MEL structures produced by each species. Certain mannose-based substances having similar properties to MEL can also be produced by Meyerozyma guilliermondii yeasts.

MEL are non-toxic and are stable at wide temperatures and pH ranges. Furthermore, MEL can be used without any additional preservatives

MEL, can be produced in more than 93 different combinations that fall under 5 main categories: MEL A, MEL B, MEL D, Tri-acetylated MEL A, and Tri-acetylated MEL B/C. These molecules can be modified, either synthetically or in nature. For example, MEL can comprise different carbon-length chains or different numbers of acetyl and/or fatty acid groups.

MEL molecules and/or modified forms thereof according to the subject invention can include, for example, tri-acylated, di-acylated, mono-acylated, tri-acetylated, di-acetylated, mono-acetylated and non-acetylated MEL, as well as stereoisomers and/or constitutional isomers thereof.

Other mannose-based substances/MEL-like substances that exhibit similar structures and similar properties, can also be used according to the subject invention, e.g., mannosyl-mannitol lipids (MML), mannosyl-arabitol lipids (MAL), and/or mannosyl-ribitol lipids (MRL).

In certain embodiments, the composition comprises a carrier. Non-limiting examples of carriers may include, for example, water; saline; physiological saline; ointments; creams; oil-water emulsions; water-in-oil emulsions; silicone-in-water emulsions; water-in-silicone emulsions; wax-in-water emulsions; water-oil-water triple emulsions; microemulsions; gels; vegetable oils; mineral oils; ester oils such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and behenyl alcohol; isoparaffins such as isooctane, isododecane (IDD) and isohexadecane; silicone oils such as cyclomethicone, dimethicone, dimethicone cross-polymer, polysiloxanes and their derivatives, preferably organomodified derivatives including PDMS, dimethicone copolyol, dimethiconols, and amodimethiconols; hydrocarbon oils such as mineral oil, petrolatum, isoeicosane and polyolefins, e.g., (hydrogenated) polyisobutene; polyols such as propylene glycol, glycerin, butylene glycol, pentylene glycol, hexylene glycol, caprylyl glycol; waxes such as beeswax, carnauba, ozokerite, microcrystalline wax, polyethylene wax, and botanical waxes; or any combinations or mixtures of the foregoing. Aqueous vehicles may include one or more solvents miscible with water, including lower alcohols, such as ethanol, isopropanol, and the like. The vehicle may comprise from about 1% to about 99% by weight of the composition, from 10% to about 85%, from 25% to 75%, or from 50% to about 65%.

Optionally, the conditioning composition can further comprise one or more other components as relevant to the specific use, including, for example, organic and/or inorganic solvents, organic and/or inorganic acids, essential oils, botanical extracts, cross-linking agents, chelators, fatty acids, alcohols, pH adjusting agents, reducing agents, buffers, enzymes, dyes, colorants, fragrances, preservatives, emulsifiers, demulsifiers, foaming agents, defoamers, bleaching agents, emollients, humectants, anti-inflammatory agents, polymers, stabilizers, silicones, thickeners, softeners, UV blockers, moisturizers, film formers, minerals, vitamins, proteins, viscosity and/or rheology modifiers, insect repellents, skin cooling compounds, skin protectants, lubricants, pearls, chromalites, micas, anti-allergenics, antimicrobials (e.g., antifungals, antivirals, antibacterials), antiseptics, pharmaceutical agents, photostabilizing agents, surface smoothers, optical diffusers, exfoliation promoters, anti-static agents, anti-wrinkling agents, wetting agents, dye transfer aids, color protectants, anti-odorants, odor capturing agents, detergents, drying agents, water repellency agents, anti-pilling agents, souring agents, starch agents, optical brightness agents, antioxidants, shrinkage control agents, starches, and mixtures thereof.

The amounts of each ingredient, whether active or inactive, are those conventionally used in cosmetics/personal care, textile processing and laundry care, to achieve their intended purpose, and typically range from about 0.0001% to about 25%, or from about 0.001% to about 20% of the composition, although the amounts may fall outside of these ranges. The nature of these ingredients and their amounts must be compatible with the production and function of the compositions of the disclosure. In preferred embodiments, the composition comprises additives that are considered dermatologically-acceptable.

In certain embodiments, the composition can include pH adjusters (e.g., citric acid, ethanolamine, sodium hydroxide, etc.) to be formulated within a wide range of pH levels. In one embodiment, the pH of the conditioning composition ranges from 1.0 to 13.0. In some embodiments, the pH of the conditioning composition ranges from 2.0 to 12.0. In some embodiments, the pH of the composition is from 3.0 to 7.0 or from 3.0 to 8.0.

The present invention can take any number of forms. These can include, for example, liquid; colloidal dispersion; micro- or nano-emulsion; gel; serum; granular, spray-dried or dry-blended powder; solid bar; concentrate; encapsulated dissolvable pod; suspension; hydrogel; multiphase solution; vesicular dispersion; foam; mousse; spray; aerosol; liquid cake; ointment; essence; paste; tablet; water soluble sheets or sachets; and/or can be impregnated into a dry or pre-moistened substrate such as a sheet (e.g., dryer sheet), ball (e.g., wool dryer ball), cloth, sponge or wipe.

### Methods of Conditioning Hair, Fibers and Textiles

In preferred embodiments, the subject invention further provides methods for conditioning hair, fibers or textiles, wherein the method comprises contacting a conditioning composition of the present invention with the hair, fibers or textiles for an effective amount of time to impart a conditioning effect thereon. In some embodiments, the composition is applied in the presence of water or another solvent. In some embodiments, the composition is rinsed from the hair, fibers or textiles after being contacted therewith.

In the context of a hair conditioner, the methods can comprise contacting the hair with the composition. In some embodiments, the composition is contacted with the hair while the hair is wet, while in other embodiments, the hair is dry. Application can comprise lathering or rubbing the composition into the strands of the hair; spraying the composition onto the hair; combing the composition through the hair; and/or other standard modes of application for hair care products.

In certain embodiments, the composition is applied to the hair simultaneously with shampooing or after shampooing the hair (i.e., after cleansing and rinsing the hair). The conditioning composition can be left in the hair as a leave-in conditioning treatment, or the composition can be rinsed after, for example, at least 15 seconds, at least 30 seconds, at least 1 minute, at least 5 minutes, to at least 60 minutes of contact.

In the context of a fiber conditioner, the composition can be contacted with fibers that are utilized in the assembly of garments and other textiles. In certain embodiments, the fibers are contacted with the composition prior to being assembled into textiles.

Chemical surfactants are often used to scour, or clean, raw fibers prior to further processing. Because scouring can be drying to the fibers, conditioning or lubrication is necessary prior to spinning, cording, and weaving the fibers into textiles. Thus, in certain embodiments, the conditioning composition can be contacted with the fibers to lubricate and soften the fibers, thereby reducing breakage, dryness, static and/or stiffness after scouring.

In the context of textile conditioning, the subject methods can be utilized during the finishing stages of textile manufacturing, as well as in household and industrial laundry. For example, in some embodiments, the conditioning composition is utilized in the form of a fabric softener that is applied to garments and other textiles during a standard wash cycle, during a drying cycle, or as a spray to wet or dry textiles.

### Production of Derivatized Cationic SLP Molecules

The subject invention provides materials and methods for producing, derivatizing and purifying sophorolipids (SLP). Advantageously, the subject invention is suitable for industrial scale production of purified SLP derivatives and uses safe and environmentally friendly materials and processes.

In preferred embodiments, the subject methods initially comprise producing standardized SLP molecular “substrates” for producing derivatized and/or purified SLP. In certain embodiments, this entails cultivating a sophorolipid-producing yeast in a submerged fermentation reactor comprising a tailored oleochemical feedstock to produce a yeast culture product, said yeast culture product comprising fermentation broth, yeast cells and SLP having a mixture of two or more molecular structures.

The mixture of molecular structures can comprise, for example, lactonic SLP, linear SLP, de-acetylated SLP, mono-acetylated SLP, di-acetylated SLP, esterified SLP, SLP with varying hydrophobic chain lengths, SLP with fatty acid-amino acid complexes attached, and others, including those that are and/or are not described within in this disclosure.

In certain embodiments, the distribution of the mixture of SLP molecules can be altered by adjusting fermentation parameters, such as, for example, feedstock, fermentation time, and dissolved oxygen levels.

As used herein “fermentation” refers to growth or cultivation of cells under controlled conditions. The growth could be aerobic or anaerobic. Unless the context requires otherwise, the phrase is intended to encompass both the growth phase and product biosynthesis phase of the process.

As used herein, a “broth,” “culture broth,” or “fermentation broth” refers to a culture medium comprising at least nutrients. If the broth is referred to after a fermentation process, the broth may comprise microbial growth byproducts and/or microbial cells as well.

The microbe growth vessel used according to the subject invention can be any fermenter or cultivation reactor for industrial use. As used herein, the term “reactor,” “bioreactor,” “fermentation reactor” or “fermentation vessel” includes a fermentation device consisting of one or more vessels and/or towers or piping arrangements. Examples of such reactor includes, but are not limited to, the Continuous Stirred Tank Reactor (CSTR), Immobilized Cell Reactor (ICR), Trickle Bed Reactor (TBR), Bubble Column, Gas Lift Fermenter, Static Mixer, or other vessel or other device suitable for gas-liquid contact. In some embodiments, the bioreactor may comprise a first growth reactor and a second fermentation reactor. As such, when referring to the addition of substrate to the bioreactor or fermentation reaction, it should be understood to include addition to either or both of these reactors where appropriate.

In one embodiment, the fermentation reactor may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, samples may be taken from the vessel for enumeration, purity measurements, SLP concentration, and/or visible oil level monitoring. For example, in one embodiment, sampling can occur every 24 hours.

The microbial inoculant according to the subject methods preferably comprises cells and/or propagules of the desired microorganism, which can be prepared using any known fermentation method. The inoculant can be pre-mixed with water and/or a liquid growth medium, if desired.

The microorganisms utilized according to the subject invention may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In preferred embodiments, the microorganism is a yeast or fungus. Examples of yeast and fungus species suitable for use according to the current invention, include, but are not limited to Starmerella spp. yeasts and/or Candida spp. yeasts, e.g., Starmerella (Candida) bombicola, Candida apicola, Candida batistae, Candida floricola, Candida riodocensis, Candida stellate and/or Candida kuoi. In a specific embodiment, the microorganism is Starmerella bombicola, e.g., strain ATCC 22214.

In certain embodiments, the cultivation method utilizes submerged fermentation in a liquid growth medium comprising a tailored oleochemical feedstock.

In one embodiment, the liquid growth medium comprises one or more sources of carbon. The carbon source can be a carbohydrate, such as glucose, dextrose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as canola oil, madhuca oil, soybean oil, rice bran oil, olive oil, corn oil, sunflower oil, sesame oil, and/or linseed oil; powdered molasses, etc. These carbon sources may be used independently or in a combination of two or more.

In preferred embodiments, the fermentation medium comprises dextrose. In another preferred embodiment, the oleochemical feedstock is tailored to include a source of oleic acid. In certain embodiments, the oleic acid content is high, e.g., at least 75%, at least 80%, at least 85%, at least 90%, at least 95%, or at least 99%. In some embodiments, the oleochemical feedstock comprises oleic acid sources exclusively.

Examples of oleic acid sources include, but are not limited to, high oleic soybean oil, high oleic sunflower oil, high oleic canola oil, olive oil, pecan oil, peanut oil, macadamia oil, grapeseed oil, sesame oil, poppyseed oil, pure oleic acid, madhuca oil, oleic acid alkyl esters, and/or triglycerides of oleic acid. In preferred embodiments, high oleic soybean oil, pure oleic acid, and/or oleic acid alkyl esters are used.

Advantageously, in certain embodiments, use of high-oleic acid and/or exclusively-oleic acid oleochemical feedstock results in a yeast culture product comprising a narrower diversity of SLP molecular structures than with feedstocks containing sources of other fatty acids, wherein the principal SLP molecules produced contain a C18 carbon chain and a single unsaturated bond at the ninth carbon. For example, in certain embodiments, greater than 50% of the SLP molecules contain an C18 carbon chain, preferably greater than 70%, more preferably greater than 85%.

In one embodiment, the liquid growth medium comprises a nitrogen source. The nitrogen source can be, for example, yeast extract, potassium nitrate, ammonium nitrate, ammonium sulfate, ammonium phosphate, ammonia, urea, and/or ammonium chloride. These nitrogen sources may be used independently or in a combination of two or more.

In one embodiment, one or more inorganic salts may also be included in the liquid growth medium. Inorganic salts can include, for example, potassium dihydrogen phosphate, monopotassium phosphate, dipotassium hydrogen phosphate, disodium hydrogen phosphate, potassium chloride, magnesium sulfate, magnesium chloride, iron sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These inorganic salts may be used independently or in a combination of two or more.

In one embodiment, growth factors and trace nutrients for microorganisms are included in the medium. Inorganic nutrients, including trace elements such as iron, zinc, copper, manganese, molybdenum and/or cobalt may also be included in the medium. Furthermore, sources of vitamins, essential amino acids, proteins and microelements can be included, for example, corn flour, peptone, yeast extract, potato extract, beef extract, soybean extract, banana peel extract, and the like, or in purified forms. Amino acids such as, for example, those useful for biosynthesis of proteins, can also be included.

The method of cultivation can further provide oxygenation to the growing culture. One embodiment utilizes slow motion of air to remove low oxygen-containing air and introduce oxygenated air. The oxygenated air may be ambient air supplemented daily through mechanisms including impellers for mechanical agitation of the liquid, and air spargers for supplying bubbles of gas to the liquid for dissolution of oxygen into the liquid.

In certain embodiments, the dissolved oxygen (DO) levels are controlled during fermentation to narrow the structural diversity of SLP molecules produced in the yeast culture product. Preferably, the DO levels are maintained at high levels such that, for example, oxygen transfer occurs at a rate at or above 50 mM, at or above 55 mM, at or above 60 mM, at or above 65 mM, or at or above 70 mM per liter per hour.

In some embodiments, the method for cultivation may further comprise adding additional acids and/or antimicrobials in the liquid medium before and/or during the cultivation process. Antimicrobial agents or antibiotics (e.g., streptomycin, oxytetracycline) are used for protecting the culture against contamination. In some embodiments, however, the metabolites produced by the yeast culture provide sufficient antimicrobial effects to prevent contamination of the culture.

In one embodiment, prior to inoculation of the reactor, the components of the liquid culture medium can optionally be sterilized. In one embodiment, sterilization of the liquid growth medium can be achieved by placing the components of the liquid culture medium in water at a temperature of about 85-100° C. In one embodiment, sterilization can be achieved by dissolving the components in 1 to 3% hydrogen peroxide in a ratio of 1:3 (w/v).

In one embodiment, the equipment used for cultivation is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Gaskets, openings, tubing and other equipment parts can be sprayed with, for example, isopropyl alcohol. Air can be sterilized by methods know in the art. For example, the ambient air can pass through at least one filter before being introduced into the vessel. In other embodiments, the medium may be pasteurized or, optionally, no heat at all added, where the use of pH and/or low water activity may be exploited to control unwanted microbial growth.

The pH of the culture should be suitable for the microorganism of interest. In some embodiments, the pH is about 2.0 to about 7.0, about 3.0 to about 5.5, about 3.25 to about 4.0, or about 3.5. Buffers, and pH regulators, such as carbonates and phosphates, may be used to stabilize pH near a preferred value. In certain embodiments, a base solution is used to adjust the pH of the culture to a favorable level, for example, a 15% to 30%, or a 20% to 25% NaOH solution. The base solution can be included in the growth medium and/or it can be fed into the fermentation reactor during cultivation to adjust the pH as needed.

In one embodiment, the method of cultivation is carried out at about 5° to about 100° C., about 15° to about 60° C. about 20° to about 45° C., about 22° to about 35° C., or about 24° to about 28° C. In one embodiment, the cultivation may be carried out continuously at a constant temperature. In another embodiment, the cultivation may be subject to changing temperatures.

According to the subject methods, the microorganisms can be cultivated in the fermentation system for a time period sufficient to achieve a desired effect, e.g., production of a desired amount of cell biomass or a desired amount of SLP. The microbial growth by-product(s) produced by microorganisms may be retained in the microorganisms and/or secreted into the growth medium. The biomass content may be, for example from 5 g/l to 180 g/l or more, from 10 g/l to 150 g/l, or from 20 g/l to 100 g/l.

In certain embodiments, fermentation of the yeast culture occurs for about 40 to 150 hours, or about 48 to 140 hours, or about 72 to 130 hours or about 96 to 120 hours. In certain specific embodiments, fermentation time ranges from 48 to 72 hours, or from 96 to 120 hours.

In some embodiments, the fermentation cycle is ended once the dextrose and/or oleic acid concentrations in the medium are exhausted (e.g., at a level of 0% to 0.5%). In some embodiments, the end of the fermentation cycle is determined to be a time point when the microorganisms have begun to consume trace amounts of SLP.

In certain embodiments, production of the SLP molecular “substrate” further comprises post-fermentation alteration of the SLP molecules produced in the yeast culture product. In one embodiment, this crude SLP composition is hydrolyzed to produce linear SLP. In some embodiments, the linear SLP are de-acetylated. In some embodiments, the linear SLP are peracetylated.

In some embodiments, the method comprises subjecting the crude SLP to alkaline hydrolysis. For example, in one embodiment, the crude SLP can be mixed with equimolar to 1.5 molar concentrations of a base solution, such as, for example, a solution of sodium hydroxide, potassium hydroxide, and/or ammonium hydroxide, to adjust the pH to, e.g., about 4 to 11, about 5 to 11, about 6 to 12, or preferably, about 7 to 9. In certain embodiments, this is achieved by treating the crude SLP with the hydroxide salt solution for 2 to 24 hours, 3 to 20 hours, or 4 to 16 hours at an elevated temperature of, e.g., 75 to 100° C., 80 to 95° C., or 85 to 90° C.

According to the subject methods, the hydrolysis process results in breakage of the lactone bond of lactonic SLP and conversion thereof to a crude linear SLP. In certain embodiments, a portion of the crude linear SLP are acetylated, di-acetylated, or peracetylated, wherein the portion comprises from, for example, 1% to 100%, 5% to 75%, or 10 to 50% of the total amount of SLP molecules. In another embodiment, a mono- or di-acetylated SLP molecule can be de-acetylated via the same alkaline hydrolysis process.

In some embodiments, when spectator cations are or may be present in the hydrolysis process, the crude linear SLP are purified using cation exchange resins. More specifically, in preferred embodiments, the crude linear sophorolipids are circulated through an ion exchange bed containing cation exchange sites using, for example, a peristaltic pump or other type of pump, for a period of time from, e.g., 15 minutes to 20 hours, 3 hours to 15 hours, 4 hours to 12 hours, or preferably, 30 minutes to 3 hours.

The amount of cation exchange sites can be, for example, equimolar to 1.5 molar the concentration of hydroxide salts used for the alkaline hydrolysis.

Advantageously, the ion exchange resins provide novel methods for purifying SLP molecules. as well as novel methods for neutralizing the pH of a reaction product without the need for standard quenching methods, which can dilute and/or change the chemical make-up of an end product.

In preferred embodiments, the linear SLP, having spectator cations removed, serve as the standardized substrates for one or more derivatization and/or purification reactions.

### Two-Step Generation of Cationic SLP Derivatives Via Aldehyde Handle

After removal of spectator cations, a two-step synthetic scheme can be employed to generate a reactive aldehyde handle on the purified linear SLP—the first isolated intermediate of the subject methods—and then install naturally-derived cationic biodegradable functional groups. Sophorolipids containing an unsaturated bond at a specific position allows for site-directed functionalization of the SLP molecule.

### Step 1—Ozonolysis

In certain embodiments, the purified linear SLP are moved to a new clean vessel containing multiple air spargers with large surface area to undergo ozonolysis. During ozonolysis of the linear SLP, the olefin moiety of the SLP molecule is converted to an ozonide, a reactive 5-membered ring.

In a preferred embodiment, the purified linear SLP are ozonated with 2 to 3 vvm of 100% ozone gas for 2 to 20 hours, 3 to 16 hours, or 4 to 10 hours. The temperature is preferably at or about −78° C.

In one exemplary embodiment, the purified linear SLP are ozonated with 3 vvm of 100% ozone gas for 4 hours. In other exemplary embodiment, the purified linear SLP are ozonated with 2 vvm of 100% ozone gas for 16 hours.

Following ozonolysis, in certain embodiments, the SLP-ozonide is degassed with compressed air for 2 to 20 hours, 3 to 16 hours, or 4 to 10 hours at 2 to 3 vvm.

In one exemplary embodiment, the SLP-ozonide is degassed with compressed air for 4 hours at 3 vvm. In another exemplary embodiment, the SLP-ozonide is degassed for 16 hours at 2 vvm.

In preferred embodiments, the SLP containing the ozonide is reduced to afford an aldehyde handle. Following degassing, the SLP-ozonide is reacted with an inorganic reducing agent selected from, for example, triphenyl phosphine, sodium borohydride, magnesium sodium bisulfite and sodium metabisulphite. In a preferred embodiment, the reducing agent is triphenyl phosphine used in equimolar concentrations to the SLP-ozonide. Afterwards, the linear SLP aldehyde is preferably brought to room temperature.

### Step 2-Reductive Amination

In preferred embodiments, step two of generating cationic SLP derivatives via the aldehyde handle comprises reductive amination of the linear SLP aldehyde.

In certain embodiments, the reductive amination comprises introducing a primary amine to the aldehyde handle under reducing conditions. This produces a stable secondary amine that serves as a covalent linkage between the SLP “scaffold” and the “cargo” of the primary amine.

First, in some embodiments, the linear SLP aldehyde is extracted with ethyl acetate from the aqueous mixture and concentrated and dried under reduced pressure (e.g., about 200 to 250 mbar, or about 240 mbar) at a temperature of about 35 to 45° C. The dried crude linear SLP aldehyde can then be dissolved in a reaction medium comprising tetrahydrofuran (THF) and/or water. The percentage of water used as the reaction medium preferably does not exceed 50% water, and typically is between 0 to 25%.

For the amination reaction, a primary amine is introduced to the extracted linear SLP aldehyde along with a reducing agent and a weak organic acid, preferably acetic acid, although other organic acids may be used (e.g., formic acid, trifluoracetic acid).

In certain embodiments, the primary amine is a cationic amino acid, such as, e.g., arginine, lysine or histidine. In certain embodiments, the primary amine is a short peptide containing repeats of cationic amino acids. In certain embodiments, the primary amine is a short peptide containing glycine residues as spacers, either between the SLP scaffold and the primary amine cargo, and/or between cationic amino acid residues.

In certain preferred embodiments, the primary amine is delivered via an amino acid ethyl ester and/or a peptide ethyl ester. In certain embodiments, the reducing agent is sodium cyanoborohydride, sodium triacetoxyborohydride or sodium borohydride.

In a preferred exemplary embodiment, the reaction utilizes an amino acid ethyl ester of arginine (Arg) with sodium triacetoxyborohydride as the reducing agent.

In another exemplary embodiment, the reaction utilizes an amino acid ethyl ester of histidine (His) or of lysine (Lys) with sodium triacetoxyborohydride as the reducing agent.

In further exemplary embodiments, the peptide ethyl esters are Arg-Arg-Arg-Arg. Gly-Gly-Arg-Arg, Gly-Arg-Gly-Arg, Gly-Arg-Arg-Arg or other combinations in which individual residues can be substituted from Arg, His, Lys, or glycine (Gly). In certain embodiments, the addition of glycine spacers enhances the water- and/or alcohol-solubility of the SLP derivative. In certain embodiments, the addition of glycine spacers enhances the antimicrobial activity of the SLP derivative by, for example, increasing the chain length of the fatty acid moiety. Advantageously, the chain length can be increased without requiring alteration of fermentation parameters during initial production of the linear SLP substrate.

### Additional or Alternative Chemical Transformations to Obtain Linear Sophorolipid Containing an Aldehyde Functional Group and a Secondary Amine.

In certain embodiments, an alternative to the two-step method utilizing ozonolysis is employed to produce linear SLP aldehydes containing a secondary amine.

In certain embodiments, the hydrolyzed linear SLP substrate serves as the starting material. In some embodiments, protecting groups can be installed on every alcohol group of the SLP sophorose ring. Non-limiting examples of protecting groups include acetyl, trimethylsilyl ether, and tert-butyldiphenylsilyl ether, but many examples of alcohol protecting groups are well known to those skilled in the art.

In preferred embodiments, the alkene group of the SLP is transformed into an aldehyde moiety without the use of ozonolysis. Instead, the alkene is epoxidized to an oxirane ring using a peracid reagent (an example of the Prilezhaev reaction) or osmium tetroxide. Examples of a peracid reagent used according to the subject method include but are not limited to m-chloroperoxybenzoic acid, peroxyacetic acid, and performic acid.

The resulting epoxide ring is then opened into a vicinal diol. In certain embodiments, this is carried out under acid-catalyzed (aqueous) or base-catalyzed (aqueous) conditions.

Lastly, the vicinal diol is oxidatively cleaved to produce the aldehyde group. Oxidative cleavage of the vicinal diol can be accomplished by an appropriate oxidant such as, for example, sodium periodate.

After the oxidative cleavage of the vicinal diol, the protecting groups, if present, may be removed by traditional methods known for a specific group. For example, silyl ether protecting groups can be removed using an aqueous source of the fluoride ion such as tetrabutylammonium fluoride, since the very strong Si—F bond that forms between the silicon and fluorine atoms drives the deprotection reaction to completion.

After obtaining the linear sophorolipid containing an aldehyde, it can be transformed into the aforementioned derivatized species through the chemical transformations outlined earlier.

In certain embodiments, it is desirable to preserve the initial alkyl chain length while carrying out chemical transformations to obtain an aldehyde functional group. This can be achieved in several ways.

In one embodiment, a two-step synthetic route is employed using a lactonic SLP as the starting material. Initially the lactonic SLP undergoes an alkaline hydrolysis to simultaneously remove acetyl groups while converting the free carboxylic acid group into a methyl ester. Using a reducing agent such as, for example, DIBAL-H, the methyl ester can be converted into an aldehyde functional group. After obtaining the linear sophorolipid containing an aldehyde, it can be transformed into the aforementioned derivatized species through the chemical transformations outlined earlier.

In another embodiment, the linear sophorolipid containing an aldehyde can be produced by a one-step, direct reduction of the lactone bond present in the lactonic SLP fermentation product. Under certain conditions, for example, with the complex reducing agent formed between diisobutylaluminum hydride and tert-butyllithium (ate complex), the lactone bond can be directly reduced in one step to the aldehyde. Besides the ate complex, other examples of possible reducing agents include but are not limited to lithium tri-tertbutoxyaluminum hydride (TBLAH), lithium diisobutyl-tert-butoxyaluminum hydride (LDBBA), and diisobutylaluminum hydride and n-butyllithium ate complex. After obtaining the linear SLP containing an aldehyde, it can be transformed into the aforementioned derivatized species through the chemical transformations outlined earlier.

### Obtaining Derivatized SLP Containing a Short-Chain or Long-Chain Amide Functional Group

In certain embodiments, the linear SLP substrate can be installed with an amide comprising cationic amino acid functional groups to produce a long-chain amide derivative (e.g., C18).

In some embodiments, the linear SLP substrate can be converted to a short-chain amide (e.g., C9) by first, truncating the fatty acid tail via oxidative cleavage, and second, installing an amide comprising cationic amino acid functional groups to the truncated acid.

Coupling agents for use in amide installation according to the subject invention can include, for example, 1-Ethyl-3-(3-dimethylaminopropyl) carbodiimide (EDCI/HOBt), Benzotriazol-1-yloxytripyrrolidinophosphonium hexafluorophosphate (PYBOP), 2-(1H-Benotriazole-1-yl)-1,1,3,3-tetramethylaminium tetrafluoroborate (TBTU), and/or N,N′-Dicyclohexylcarbodiimide/1-Hydroxybenzotriazole (DCC/HOBt). In certain embodiments, the preferred coupling agent is EDCI/HOBt.

In certain embodiments, the linear SLP comprising the aldehyde handle as described previously can be converted into a long-chain or short-chain amide utilizing similar reaction schemes. In some embodiments, the truncated acid can serve as a substrate for installing the aldehyde handle described previously.

### Ion Exchange/Purification

In certain embodiments, the unique cationic nature of the SLP derivatives of the subject invention allows for cationic ion exchange resins to be used for selective purification, e.g., selective retention of cationic species and/or selective removal of unreacted SLP and SLP that did not contain the desired carbon chain length or character. Thus, in certain embodiments, the subject invention provides novel methods of purifying SLP and SLP derivatives using cationic ion exchange resins.

In certain embodiments, following reductive amination, the cationic SLP derivative can be extracted from the reductive amination reaction mixture via a standard liquid-liquid extraction using an organic solvent, preferably ethyl acetate, washed with a pH 9.0 sodium carbonate buffer, and concentrated under reduced pressure (e.g., about 200 to 250 mbar, or about 240 mbar). The mixture can then be resuspended in deionized water and purified using cationic exchange resins.

In certain embodiments, the extracted cationic SLP are circulated through an ion exchange bed containing equimolar to 1.5 molar amounts of cation exchange sites to the concentration of the crude linear cationic SLP with, for example, a peristaltic pump or other type of pump, for a period of 2 to 20 hours, 3 to 15 hours, or 4 to 12 hours.

In preferred embodiments, removal of the SLP cationic derivatives from the resin is accomplished by application of an electrolyte solution containing a large concentration of monovalent metallic cations, wherein the large concentration is 1.5 to 15 molar equivalents, or 2 to 10 molar equivalents to the concentration of the SLP cationic derivatives. The monovalent metallic cations in large concentration outcompete the bound SLP cationic derivatives, allowing for them to exchange on the resin and produce a highly purified stream of SLP cationic derivatives.

In some embodiments, following reductive amination, the cationic SLP derivative can be purified by stirring it with saturated ammonium chloride solution to produce a stirred mixture; extracting the cationic SLP derivative by applying CH₂Cl₂ solvent (3×) to the stirred mixture to produce an extraction mixture; removing trace water from the extraction mixture by applying MgSO₄ or Na₂SO₄; drying the extraction mixture under elevated pressure (e.g., 350 to 450 mbar, or 400 mbar) and at 35 to 45° C. to remove the CH₂Cl₂ solvent; and, applying 21% NaOEt/EtOH solution, NaHCO₃ or KHCO₃ base in ethanol to remove acetyl R groups from the cationic SLP derivative. The de-acetylated linear 10 cationic SLP derivative can then be converted to an HCl salt via reaction with a 1.25M HCl/EtOH solution.

1. A conditioning composition comprising a cationic sophorolipid (SLP) molecule that has been derivatized to have one or the following formulas: 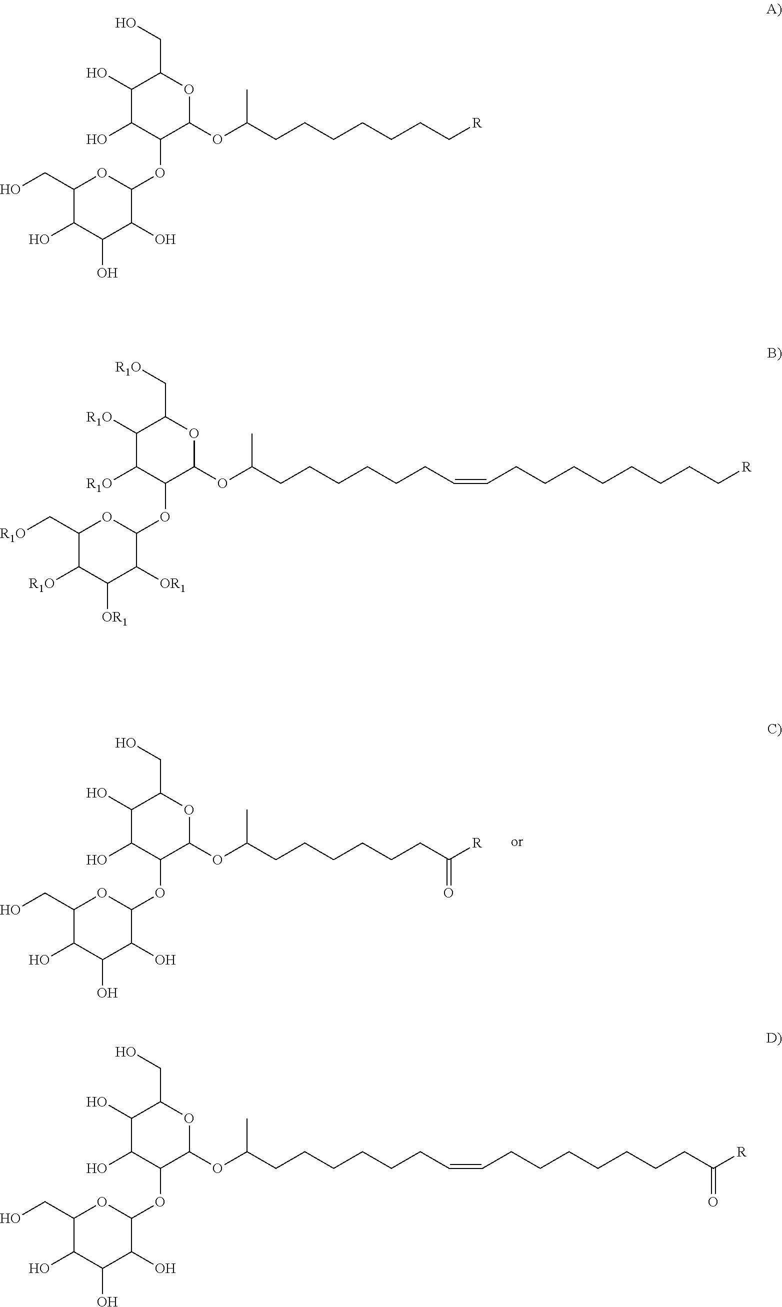
wherein R=arginine, lysine, or histidine, or a peptide comprising repeats thereof and R₁=H, acetyl, butyl, or isobutyl.

2. The composition of claim 1, further comprising a non-derivatized SLP molecule.

3. The composition of claim 2, wherein the ratio of the cationic derivatized SLP to non-derivatized SLP is from 1:10 to 1:1000.

4. The composition of claim 1, further comprising a mannosylerythritol lipid (MEL).

5. The composition of claim 4, wherein the ratio of the cationic derivatized SLP to MEL is from 1:10 to 1:1000.

6. The composition of claim 4, wherein the ratio of the cationic derivatized SLP to MEL is from 1000:1 to 10:1.

7. The composition of claim 1, further comprising one or more other components selected from the group consisting of: carriers, organic and/or inorganic solvents, organic and/or inorganic acids, essential oils, botanical extracts, cross-linking agents, chelators, fatty acids, alcohols, pH adjusting agents, reducing agents, buffers, enzymes, dyes, colorants, fragrances, preservatives, emulsifiers, demulsifiers, foaming agents, defoamers, bleaching agents, emollients, humectants, anti-inflammatory agents, polymers, stabilizers, silicones, thickeners, softeners, UV blockers, moisturizers, film formers, minerals, vitamins, proteins, viscosity and/or rheology modifiers, insect repellents, skin cooling compounds, skin protectants, lubricants, pearls, chromalites, micas, anti-allergenics, antimicrobials, antifungals, antivirals, antibacterials, antiseptics, pharmaceutical agents, photostabilizing agents, surface smoothers, optical diffusers, exfoliation promoters, anti-static agents, anti-wrinkling agents, wetting agents, dye transfer aids, color protectants, anti-odorants, odor capturing agents, detergents, drying agents, water repellency agents, anti-pilling agents, souring agents, starch agents, optical brightness agents, antioxidants, shrinkage control agents, starches, and mixtures thereof.

8. The composition of claim 1, formulated as a liquid; colloidal dispersion; micro- or nano-emulsion; gel; serum; granular, spray-dried or dry-blended powder; solid bar; concentrate; encapsulated dissolvable pod; suspension; hydrogel; multiphase solution; vesicular dispersion; foam; mousse; spray; aerosol; liquid cake; ointment; essence; paste; tablet; water soluble sheet or sachet; and/or can be impregnated into a dry or pre-moistened substrate such as a sheet, ball, cloth, sponge or wipe.

9. A method for conditioning hair, a fiber or a textile, the method comprising contacting a conditioning composition comprising a cationic derivatized sophorolipid (SLP) molecule with the hair, fiber or textile for an effective amount of time to achieve a desired level of conditioning, wherein the derivatized SLP molecule has one of the following formulas: 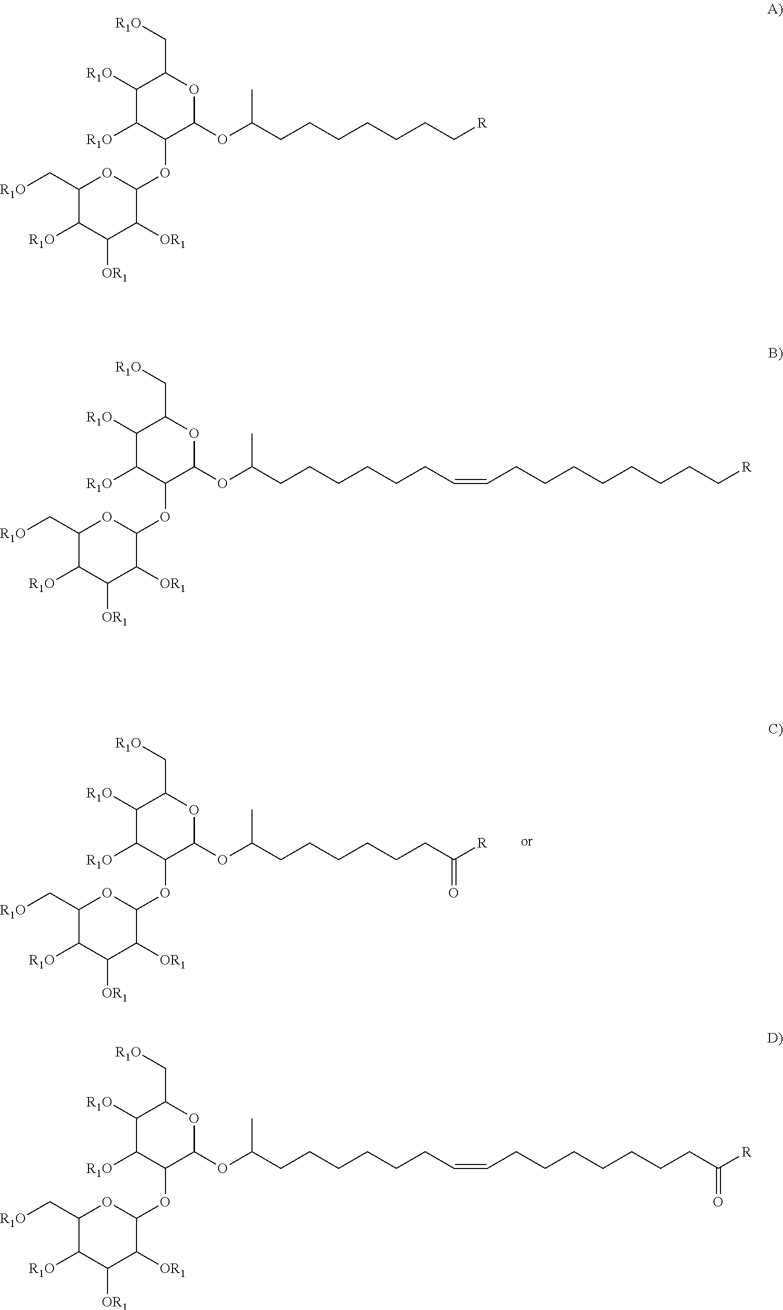
wherein R=arginine, lysine, glycine, or histidine, or a peptide comprising repeats thereof and R₁=H, acetyl, butyl, or isobutyl

10. The method of claim 9, further comprising contacting a non-derivatized SLP molecule with the hair, fiber or textile.

11. The method of claim 10, wherein the ratio of the derivatized cationic SLP to non-derivatized SLP is from 1:10 to 1:1000.

12. The method of claim 9, further comprising contacting a mannosylerythritol lipid (MEL) with the hair, fiber or textile.

13. The method of claim 12, wherein the ratio of the derivatized cationic SLP to MEL is from 1:10 to 1:1000.

14. The method of claim 12, wherein the ratio of the derivatized cationic SLP to MEL is from 1000:1 to 10:1.

15. The method of claim 9, wherein the conditioning composition further comprises one or more other components selected from the group consisting of: carriers, organic and/or inorganic solvents, organic and/or inorganic acids, essential oils, botanical extracts, cross-linking agents, chelators, fatty acids, alcohols, pH adjusting agents, reducing agents, buffers, enzymes, dyes, colorants, fragrances, preservatives, emulsifiers, demulsifiers, foaming agents, defoamers, bleaching agents, emollients, humectants, anti-inflammatory agents, polymers, stabilizers, silicones, thickeners, softeners, UV blockers, moisturizers, film formers, minerals, vitamins, proteins, viscosity and/or rheology modifiers, insect repellents, skin cooling compounds, skin protectants, lubricants, pearls, chromalites, micas, anti-allergenics, antimicrobials, antifungals, antivirals, antibacterials, antiseptics, pharmaceutical agents, photostabilizing agents, surface smoothers, optical diffusers, exfoliation promoters, anti-static agents, anti-wrinkling agents, wetting agents, dye transfer aids, color protectants, anti-odorants, odor capturing agents, detergents, drying agents, water repellency agents, anti-pilling agents, souring agents, starch agents, optical brightness agents, antioxidants, shrinkage control agents, starches, and mixtures thereof.

16. The method of claim 9, wherein the conditioning composition is rinsed from the hair, fiber or textile with water or other solvent after the effective amount of contact time to achieve conditioning.

17. The method of claim 9, wherein the conditioning composition is left on the hair, fiber or textile as a leave-in conditioner.

18. The method of claim 9, wherein the method imparts one or more of the following benefits upon the hair, fiber or textile: reducing static and/or frizz; increasing strength; reducing breakage; reducing tangling and matting; improving shine and softness; increasing oil and/or moisture retention; reducing wrinkles; and/or imparting a fragrance.

19. A method of conditioning hair, a fiber or a textile, the method comprising contacting a composition of claim 1 with the hair, fiber or textile.

20. (canceled)

21. The method of claim 19, wherein the conditioning composition is left on the hair, fiber or textile as a leave-in conditioner.
